(19) United States
(12) Patent Application Publication
(10) Pub. No.: US 2008/0306473 A1
(43) Pub. Date: Dec. 11, 2008

(54) Tissue-treating device with medium-control mechanism

(76) Inventor: Georg Schuele, Menlo Park, CA (US)

Correspondence Address: MINTZ, LEVIN, COHN, FERRIS, GLOVSKY AND POPEO, P.C;ATTN: PATENT INTAKE CUSTOMER NO. 30623, ONE FINANCIAL CENTER, BOSTON, MA, 02111, US

(21) Appl. No.: 11/999,993

(22) Filed: Dec. 7, 2007

Related U.S. Application Data
(60) Provisional application No. 60/873,270, filed on Dec. 7, 2006.

(51) Int. Cl. A61B 18/20

(52) U.S. Cl. 606/16

(57) ABSTRACT

Disclosed is a tissue-treating device. The device includes a waveguide to guide light, and a medium-control mechanism to change a medium disposed near an area of a tissue to which light from the waveguide is applied. The medium-control mechanism to change the medium may be configured to change the medium such that light energy applied to the area of the tissue with the changed medium results in changes to the area of the tissue that are different from changes to the area of the tissue resulting from application of light energy to the area without changing the medium.

### CROSS-REFERENCE TO RELATED APPLICATIONS

This application claims priority to provisional U.S. application Ser. No. 60/873,270, entitled “Control of laser tissue interaction and efficacy enhancement”, and filed Dec. 7, 2006, the content of which is hereby incorporated by reference in its entirety.

### BACKGROUND

The present disclosure relates to a tissue-treating device, and more particularly to a tissue-treating device (e.g., a laser device) with a medium-control mechanism to control the interaction between the tissue-treating device and a target area of the tissue.

Devices employing fibers to deliver light (e.g., laser light) to an area of a tissue are widely used in laser medicine. Generally, the laser energy delivered to the treated tissue is controlled by the controlling the parameters of the laser energy (e.g., amplitude and frequency). Variations to the wavelength, peak intensity and the duration (e.g., period) of the generated laser light applied can thus result in different tissue effects. For example continuous application of laser light at low power settings lead to a thermal coagulation of tissues, but short pulsed high peak lasers burst may lead to tissue ablation.

### SUMMARY

Disclosed herein are methods and devices to control the light/tissue interactions by controlling the environment of the tissue site to which the light (e.g., laser light) is applied, thus avoiding having to directly control the operation parameters of the light and/or light source (e.g., changing, for example, a laser's power level and/or frequency setting).

In one aspect, a tissue-treating device is disclosed. The device includes a waveguide to guide light, and a medium-control mechanism to change a medium disposed near an area of a tissue to which light from the waveguide is applied.

Embodiments of the tissue-treating device may include one or more of the following features.

The medium-control mechanism to change the medium may be configured to change the medium such that light energy applied to the area of the tissue with the changed medium results in changes to the area of the tissue that are different from changes to the area of the tissue resulting from application of light energy to the area without changing the medium.

The device may further include a laser generating module to generate laser light. The waveguide may be a laser fiber.

The medium-control mechanism may include a conduit to deliver substances to the area of the tissue. The delivered substances may cause the medium disposed near the area of the tissue to be displaced. The conduit to deliver the substances may be configured to deliver one or more of, for example, a gas and/or a fluid. The gas may include air. The substances may include substances having low-light absorption properties. The substances may include particles adapted to increase tissue ablation by mechanical force. The particles adapted to increase tissue ablation by mechanical force may include sapphire particles.

The medium-control mechanism may further include a controller to regulate the level of the substances delivered through the conduit from a substance source. The device may further include the substance source. The controller to regulate the level of the substances delivered through the conduit may be configured to periodically adjust the level of the delivered substances. The light may be pulsating laser and the controller may be configured to synchronize the level of the delivered substances to pulses of the laser.

The device may further include a reflector to direct the light exiting the waveguide to the area of the tissue.

In another aspect, a method for treating tissue is disclosed. The method includes changing a medium disposed near an area of the tissue, and applying light to the area of the tissue.

Embodiments of the method may include any of the features described herein in relation to the device and may also include one or more of the following features.

Changing the medium may include displacing the medium with substances delivered through a conduit.

Displacing the medium with the substances delivered through the conduit may include regulating the level of the substances delivered through the conduit. Regulating the level of the substances delivered through the conduit may include periodically adjusting the level of the delivered substances.

Applying the light to the area of the tissue may include applying pulsating laser to the area of the tissue. Regulating the level of the substances delivered through the conduit may include synchronizing the level of the substances to pulses of the laser.

Applying the light to the area of the tissue may include applying laser light to the area of the tissue. Applying the laser light may further include generating the laser light, directing the generated light through a waveguide, and reflecting the light exiting the waveguide to the area of the tissue.

The methods and devices described herein may be used, for example, in the treatment of Benign Prostatic Hyperplasia (BPH) to ablate the prostate tissue by delivering laser energy through a fiber to the prostate tissue, in the treatment of stone removal from various organs/tissues (e.g., removal of urinary and gall bladder stones), etc.

Details of one or more implementations are set forth in the accompanying drawings and in the description below. Further features, aspects, and advantages will become apparent from the description, the drawings, and the claims.

### BRIEF DESCRIPTION OF THE DRAWINGS

FIG. 1 is a cross-sectional view of an exemplary tissue-treating device.

FIG. 2 is a cross-sectional view of another exemplary tissue-treating device.

FIG. 3 is a flow chart of an exemplary tissue treating procedure.

FIG. 4 is a photograph of a tissue treated with laser energy.

Like reference numbers and designations in the various drawings indicate like elements.

### DETAILED DESCRIPTION

Described herein is a method, apparatus and computer program product to change a medium disposed near an area to be treated of a tissue and applying light energy, such as laser energy, to the area to be treated. Changing the medium by, for example, displacing the medium to control how much of the light energy gets absorbed by the medium, is performed such that light applied to the area of the tissue with the changed medium results in changes to the area of the tissue that are different from changes to the area of the tissue resulting from application of light to the area of the tissue without changing the medium.

Thus, in some embodiments, the light-tissue interaction of light (delivered, for example, via an optical fiber, or laser fiber) with the tissue can be controlled, and the efficacy of the laser-light tissue treatment enhanced, by controlling the environment in which a light-based treatment device operates.

Use of laser energy to treat tissue (e.g., in the treatment of Benign Prostatic Hyperplasia (BPH) a free running pulsed Holmium laser, generating light with a wavelength of 2.1 μm, is used to ablate the prostate tissue by delivering laser energy through a fiber to the prostate tissue) is motivated by the fact that much of the laser light energy tends to be absorbed by water. Because tissue is composed of, on average, of 85-95% of water, a significant portion of the laser energy is absorbed by the tissue. Consequently, substantial heating at the irradiated site results. In a typical clinical setting, the tissue to be treated is immersed in an environment (e.g., blood) of which water is a significant constituent. Furthermore, in some embodiments, the tissue treating device includes an endoscope having a rinsing mechanism to clean the area of the tissue being treated from any debris (e.g., tissue remnants) produced, for example, as a result of the treatment. Thus, under those circumstances, the area being treated of the tissue will be surrounded by, for example, water, or other rinsing or flushing agents.

To avoid substantial absorption of the applied light energy by the water-based interface (i.e., medium) disposed between the waveguide tip (through which light is emitted) and the tissue, the waveguide (e.g., an optical fiber) generally has to be brought in close contact with the tissue. Alternatively, the proportion of the energy delivered to the tissue can be increased if there is less absorption by the medium surrounding the tissue.

Accordingly, the level of energy delivered to the tissue (e.g., to perform clinical procedures such as ablation and/or coagulation) can be regulated by controlling the medium disposed near, or surrounding, the area to be treated of the tissue.

Control of the medium (e.g., by changing it in some manner) has additional effects. For example, changing the medium disposed near the target area to be treated of the tissue causes the thermal or mechanical properties of the medium to change, which in turn enables control of the coagulation efficiencies. Other properties and characteristics of the laser-tissue interactions can be controlled by controlling the interfacing medium disposed near the tissue. For example, by changing the optical characteristics of the medium (e.g., absorbing and/or scattering characteristics) an increased level of energy and more focused light (laser) beam can reach the tissue. Thus, changing of the medium's optical characteristics enables establishing a more effective “optical chain” from the source to the tissue to perform faster/better tissue ablation.

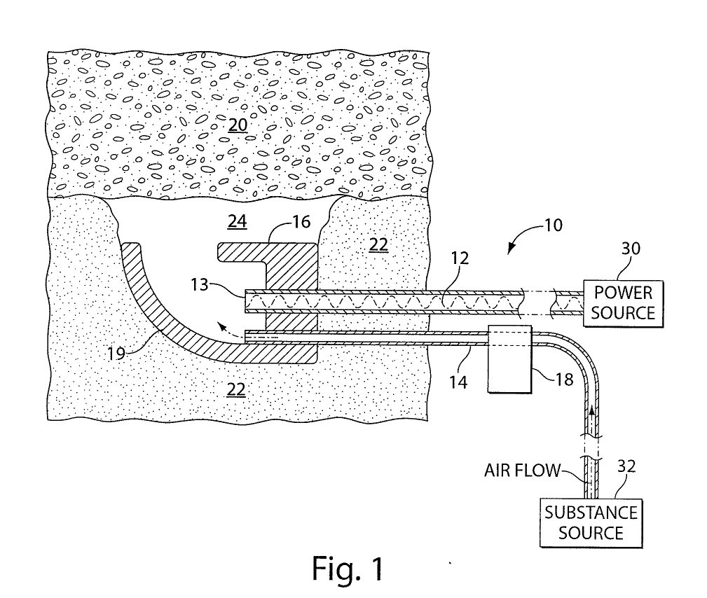

Referring to FIG. 1, a cross-sectional view of an exemplary tissue-treating device 10 is shown. To treat an area of the tissue 20, the device 10 is generally placed proximate to the tissue. Under some circumstances, direct contact between the device 10 (also referred to as an applicator) and the area of the tissue 20 may be required, while in other circumstances such contact may be avoided (e.g., to reduce the likelihood of injuring the tissue). The tissue being treated includes human tissues as prostate tissue and/or other types of tissue on which light-based treatment has therapeutic benefits. Disposed near, or surrounding, the tissue is the medium material 22, for example, a water-based medium such as blood and/or water-based rinsing agents to rinse the tissue. When the device 10 is placed near the tissue 20 the medium 22 separates the device 10 from the tissue 20 and, in effect, interfaces the device 10 and the tissue 20. The device-tissue interaction resulting from application of light energy discharged from the device 10 will thus be effected by the medium 22 (e.g., the medium 22 may absorb at least some of the light energy being directed to the tissue).

The tissue-treatment device 10 includes a waveguide 12 to guide light that is to be applied to the targeted area of the tissue 20. In some embodiments, the waveguide is an optical fiber configured to guide light energy of particular frequencies through it. For example, when holmium laser is applied, an appropriate fiber with minimal energy loses (absorption) for the laser's particular wavelength is used. Suitable fibers to transmit holmium laser light may include low OH silica fiber. In some embodiments, a hollow fiber may be used to transmit light having infrared (IR) wavelengths (light with such wavelength may be generated, for example, using a CO₂ laser). In circumstances in which a hollow fiber is used, the internal hollowed portion of the fiber may also be used to deliver medium-changing substances (i.e., substances to change the medium, as discussed in more details below). Light exiting from a tip 13 of the waveguide 12 is directed to the area to be treated of the tissue 20. The waveguide 12 is secured to a head piece 16 that holds the tip 13 of the waveguide 12 substantially in place so as to avoid excessive wobbling of the waveguide 12. A power/light source 30 to generate light is coupled to the other end of the waveguide 12 through a suitable adapter or interface. The light source may be, in some embodiments, a laser light source, such as holmium laser generating laser light having a wavelength of 2.1 μm and with peak powers of 5 kW. Other suitable laser sources include Tulium lasers, CO₂ lasers, etc. In some embodiments, the waveguide may include a fiber laser, such as an Er:YAG fiber laser, in which the fiber generates the laser internally (typically, the laser light is generated at a section of the fiber that may be outside of a patient's body), and the generated laser light is then transmitted via the rest of the fiber to be discharged near the target area of the tissue. Other types of lights source may be used, including, for example, white light source fitted with suitable optical filter. In some embodiments, the light source may also be used in the implementation of a visualization mechanism (e.g., generating an aiming beam so that an unseen treating laser light, such as a Helium-Neon laser, could be properly aimed at the treatment site) and/or to provide illumination of the treatment site.

In some embodiments, the waveguide 12 includes a laser fiber that acts as a gain medium for the laser system and transfers the generated laser energy to the tip 13 to be discharged and directed to the area to be treated of the tissue 20.

The device 10 also includes a medium control-mechanism to change the medium disposed near the area to be treated of the tissue 20. In some embodiments, the medium-control mechanism includes a conduit 14, such as a hollow pipe or tube, to deliver medium-changing substances (also referred to as irrigation substances) that, when the substances interact with the medium 22, cause changes to the medium. For example, in some embodiments, the conduit 14 delivers gases or fluids, such as air that cause regions of the medium 22 to be displaced.

More particularly, and as shown in FIG. 1, when irrigation substances such as air are delivered through the conduit 14 and released into the medium, the air pushes the medium, causing portions of the medium to be displaced. Consequently, an air pocket 24, extending from the edge of the tissue 20 to the inner walls of the head piece 16 (i.e., near the ends of the waveguide 12 and the conduit 14) is formed. As a result of the formation of the pocket 24, light energy can reach the tissue 20 with a smaller portion of the light being absorbed (and thus lost) by the medium 22. Other types of gases and/or fluids may be delivered by the conduit 14 to cause displacement of the medium 22, and thus reduce the proportion of light energy that would otherwise be absorbed by the medium 22.

In some embodiments, the substances released into the medium 22 cause physical and/or chemical changes of the properties of the medium 22. For example, the substances delivered by the conduit 14 can include substances having low-light absorption properties. Thus, when released into the medium 22, such low-light absorption substances mix into the medium 22 and cause a general change (at least in the region of the medium 22 into which molecules of the delivered substances mix) to the light absorption properties of the medium 22, e.g., lower the light-absorption property of the medium. Consequently, as a result of the release of such a medium-changing substances into the medium 22, less of the light energy directed from the tip 13 of the waveguide 12 will be absorbed by the medium 22, and thus a higher proportion of the available light energy discharged from the waveguide 12 will be applied to the area of the tissue 20. In some embodiments, the substances released include bubbles (e.g., air bubbles) that facilitate the ablation process. Particularly, the bubbles promote the “cavitation” effect in which the bubbles create shock waves to accelerate particles to achieve improved tissue ablation.

In some embodiments, the medium-changing substances delivered through the conduit 14 and released into the medium 22 include particles, such as sapphire particles, that are adapted to increase tissue ablation by mechanical force. Such particles can absorb light energy which causes them to accelerate toward the target area of the tissue to achieve a stronger ablation effect. These particles may promote different types of interaction mechanisms that depend on particle sizes. For example, small particles may cause “micro-explosion” type interaction.

The medium-control mechanism further includes a controller 18, coupled to the medium-changing substance source 32, to regulate the level (i.e., the quantity) of the substances delivered from the substance source 32 and through the conduit 14 to thus control the device-tissue interaction. Particularly, by regulating (e.g., temporal regulation) the level of substances discharged into the medium 22, the energy applied to the tissue 20 can be regulated. In some embodiments, the controller 18 may be configured to periodically adjust the level of the delivered substances to implement a substance delivery cycle (i.e., substances released to the medium during an ON stage of the cycle, and withheld during the OFF stage of the cycle). Such a regulation mechanism could be used to, for example, vary the level of energy applied to the area of the tissue 20 (e.g., where the medical treatment requires such a periodic application of energy) without having to vary the actual power level of the light source.

In some embodiments, the controller 18 is configured to synchronize delivery of the medium-changing substances to pulses of a pulsating light source (e.g., a pulsating laser). For example, when a pulsating light source is used, it may be wasteful to release medium-changing substances into the medium 22 while the light source is in its OFF stage. Accordingly, the controller 18 could be configured so that flow of the medium-changing substances is ceased while light energy is not being guided discharged through the tip 13 of the waveguide 12. The controller would cause the flow of substances to commence or resume when discharge of light energy.

The controller 18 may be implemented, in some embodiments, using a valve mechanism (not shown) to turn on and off the flow of substances through the conduit 14. The valve mechanism could be actuated using a suitable actuation mechanism (not shown) such as a pneumatic actuator, and electromechanical actuator, etc. The actuator mechanism could, in turn, be controlled by, for example, a processor-based device (not shown). Such a processor-based device can receive control input data (e.g., actuation period) through a user-interface disposed on the controller 18, or through remote electronic transmission of the data. Based on the input data, the processor could generate control signals to control the actuation mechanism and thus to actuate the valve mechanism. The controller 18 may also include pre-defined operation profiles, for example, a temporal profile specifying how the valve is to be actuated to turn flow of substances through the conduit 14 on and off. The processor-based device may include a computer and/or other types of processor-based devices suitable for multiple applications. Such devices can include volatile and non-volatile memory elements, and peripheral devices to enable input/output functionality. Such peripheral devices include, for example, a CD-ROM drive and/or floppy drive, or a network connection, for downloading related content. Such peripheral devices may also be used for downloading software containing computer instructions to enable general operation of the controller 18, and for downloading software implemented programs to perform operations to control, for example, operation of the valve mechanism to thus enable regulating the level of the substances delivered through the conduit 14 and released into the medium 22.

As further shown in FIG. 1, in some embodiments, the device 10 may include a reflector 19 that is attached to, or integrally extending from, the head piece 16. Use of a reflector to reflect light discharged from the tip 13 of the waveguide 12 enables optical control of the direction of the light. For example, the reflector 19 may have a structure, e.g., parabolic, structure, that enable focusing the light energy at the target area of the tissue 20. The reflector 19 may also enable deflection of the medium-changing substances so that the substances concentrate in particular area between the device 10 and the tissue 20.

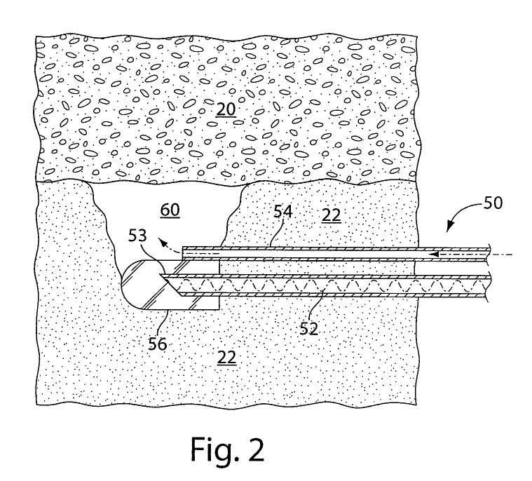

Referring to FIG. 2, a cross-sectional view of another exemplary tissue-treating device 50 is shown. The device 50 includes a waveguide 52 (e.g., a laser fiber), which may be similar to the waveguide 12 shown in FIG. 1. The waveguide 52 is coupled to a light source or a power source (e.g., a power source to pump energy into the laser fiber to direct laser light through the laser fiber 52). Like the waveguide 12, the waveguide 52 also includes a tip 53 through which the light energy is discharged.

As shown, the region of the waveguide 52 around the tip 52 is encased in a glass housing 56. The tip 53 of the waveguide 52 is generally diagonally cut so that the tip has an angled (e.g., tapered) end. The glass housing 56 is mounted in a manner that forms an air pocket adjacent the angled face. This air pocket defines a medium adjacent the tip of the waveguide which has an index of refraction sufficiently different from the index of refraction of the optical fiber that total internal reflection can take place at the angled face. The tip's angled face is thus configured to reflect light traveling down the waveguide in a direction transverse to the longitudinal axis of the waveguide. Particularly, the angled face of the tip is configured to cause the light to undergo total internal reflection. Further details regarding the total internal reflection mechanism that may be implemented using a fiber waveguide and a glass housing are provided, for example, in U.S. Pat. No. 5,772,657, entitled “Side firing fiber optic laser probe”, the content of which is hereby incorporated by reference in its entirety.

The device 50 includes a medium-control mechanism that includes, in some embodiments, a conduit 54 such as a pipe or a tube. Like the conduit 14 of the device 10, the conduit 54 is configured to deliver medium-changing substances that change the medium 22. For example, the delivered substances could include air which causes, when released into the medium 22, to displace regions of the medium 22 and thus create an air pocket 60. Once the air pocket 60 is formed, light can pass through the glass housing 56 and be directed through the air pocket 60 to the area to be treated of the tissue 60. In some embodiments, the conduit 54 is configured to deliver other types of medium-changing substances that cause changes to physical/chemical properties of the medium 22.

The tissue-treating device 50 may include additional modules and units similar in structure and functionality to those additional modules and units described in relation to the device 10 shown in FIG. 1.

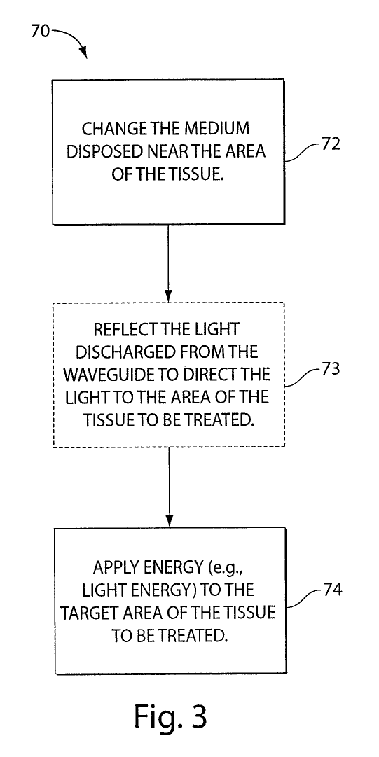

Referring to FIG. 3, a flow chart of an exemplary tissue treating procedure 70 is shown. A tissue-treating device, such as the device 10 or the device 50, is placed near the target area to be treated of the tissue 20. The area to be treated is typically surrounded by a medium, e.g., a water-based medium such as blood. To control the level and manner of energy applied to the area to be treated, the device's medium-control mechanism is used to change 72 the medium disposed near the area of the tissue. Changing the medium can be performed, for example, by causing regions of the medium (e.g., the medium 22 shown in FIGS. 1 and 2) to be displaced. Displacement of the medium is achieved by, for example, delivering air from an air source through a conduit and releasing it near the region of the medium disposed near the area to be treated so that the medium is displaced and an air pocket is formed. Alternatively and/or additionally, other substances may be delivered through the conduit and released into the medium to otherwise change the medium. For example, substances that cause the light-absorption properties of the medium to change so that the medium absorbs less light may be released into the medium. The delivery of medium-changing substance through the conduit of the medium-control mechanism may be regulated using a controller. For example, a controller that includes a valve mechanism and a microprocessor that controls an actuator to actuate the valve mechanism may be used to control the flow of the medium-changing substances in the conduit.

Having changed the medium, light energy is applied 74 to the target area of the tissue to be treated. The applied energy may be used, for example, to ablate the tissue. The light energy may be laser light energy generated and delivered by, for example, a power source and laser fiber. In some embodiments, the laser light generated is pulsating light and thus, to improve efficiency of the tissue-treating procedure, the changing of the medium operation is synchronized to the pulsating application of laser energy to the tissue. For example, release of medium-changing substances into the medium is synchronized to the beginning of laser pulses.

Optionally, in some embodiments, the light discharged from the tip of the waveguide is reflected 73 to direct the light to the area to be treated of the tissue 20.

Controlling the flow of medium-changing substances to change the medium near the area to be treated of the tissue can achieve different tissue effects and/or enable control of the ablation speed (in circumstance where tissue ablation is to be performed). Control of the flow of medium-changing substances (including substances that also impart a therapeutic effect) can also result in improved haemostasis and/or tissue coagulation.

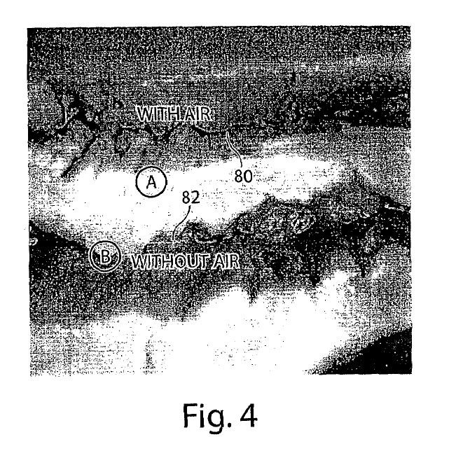

Referring to FIG. 4, a photograph comparing the effects of performing laser-based tissue-treatment procedure with and without the use of medium-changing substances is shown. The two ablated lines 80 and 82 were generated by the same device in which the same energy and laser settings and same fiber movement speeds were used. In the case of upper ablation line 80, air was released into the medium, whereas with respect to lower ablation line 82 no medium-changing substance was released into the medium. The experiment was performed in a water bath (i.e., the medium surrounding the tissue was water). As shown, the ablation area near the ablation line 80 (i.e., the ablation area resulting from performing the tissue-treating procedure with air released into the medium) showed a much more defined area of tissue necrosis where thermal coagulation occurred (area A) compared to the tissue area B (i.e., the ablation area resulting from performing the tissue-treating procedure without air being released into the medium). Thus, the use of air to displace the water resulted in more of the applied energy being absorbed by the tissue than was the case when the medium was not displaced (i.e., in area B) or otherwise changed. Additionally, the ablation depths and the ablated volumes were different for the two areas with the area A having more extensive ablation depth and volume than area B.

A number of embodiments of the invention have been described. Nevertheless, it will be understood that various modifications may be made without departing from the spirit and scope of the invention. Accordingly, other embodiments are within the scope of the following claims.

What is claimed is:

1. A tissue-treating device comprising:
a waveguide to guide light; and
a medium-control mechanism to change a medium disposed near an area of a tissue to which light from the waveguide is applied.

2. The device of claim 1 wherein the medium-control mechanism to change the medium is configured to change the medium such that light energy applied to the area of the tissue with the changed medium results in changes to the area of the tissue that are different from changes to the area of the tissue resulting from application of light energy to the area without changing the medium.

3. The device of claim 1 further comprising:
a laser generating module to generate laser light, wherein the waveguide is a laser fiber.

4. The device of claim 1 wherein the medium-control mechanism comprises a conduit to deliver substances to the area of the tissue.

5. The device of claim 4 wherein the delivered substances cause the medium disposed near the area of the tissue to be displaced.

6. The device of claim 4 wherein the conduit to deliver the substances is configured to deliver one or more of: a gas and a fluid.

7. The device of claim 6 wherein the gas includes air.

8. The device of claim 4 wherein the substances include substances having low-light absorption properties.

9. The device of claim 4 wherein the substances include particles adapted to increase tissue ablation by mechanical force.

10. The device of claim 9 wherein the particles include sapphire particles.

11. The device of claim 4 wherein the medium-control mechanism further comprising a controller to regulate the level of the substances delivered through the conduit from a substance source.

12. The device of claim 11 further comprising the substance source.

13. The device of claim 11 wherein the controller to regulate the level of the substances delivered through the conduit is configured to periodically adjust the level of the delivered substances.

14. The device of claim 11 wherein the light is pulsating laser and wherein the controller is configured to synchronize the level of the delivered substances to pulses of the laser.

15. The device of claim 1 further comprising:
a reflector to direct the light exiting the waveguide to the area of the tissue.

16. A method for treating tissue, the method comprising:
changing a medium disposed near an area of the tissue; and
applying light to the area of the tissue.

17. The method of claim 16 wherein changing the medium comprises changing the medium such that light applied to the area of the tissue with the changed medium results in changes to the area of the tissue that are different from changes to the area of the tissue resulting from application of light to the area without changing the medium.

18. The method of claim 16 wherein changing the medium comprises:
displacing the medium with substances delivered through a conduit.

19. The method of claim 18 wherein displacing the medium with the substances delivered through the conduit comprises displacing the medium with one or more of: a gas and a fluid.

20. The method of claim 19 wherein the gas includes air.

21. The method of claim 18 wherein the substances include substances having low-light absorption properties.

22. The method of claim 18 wherein the substances include particles adapted to increase tissue ablation by mechanical force.

23. The method of claim 18 wherein displacing the medium with the substances delivered through the conduit comprises regulating the level of the substances delivered through the conduit.

24. The method of claim 23 wherein regulating the level of the substances delivered through the conduit comprises periodically adjusting the level of the delivered substances.

25. The method of claim 23 wherein applying the light to the area of the tissue comprises applying pulsating laser to the area of the tissue, and wherein regulating the level of the substances delivered through the conduit comprises synchronizing the level of the substances to pulses of the laser.

26. The method of claim 16 wherein applying the light to the area of the tissue comprises applying laser light to the area of the tissue.

27. The method of claim 26 wherein applying the laser light further comprises:
generating the laser light;
directing the generated light through a waveguide; and
reflecting the light exiting the waveguide to the area of the tissue.
